(19) United States
(12) Patent Application Publication
(10) Pub. No.: US 2022/0233539 A1
(43) Pub. Date: Jul. 28, 2022

(54) COMPOSITIONS AND METHODS FOR INHIBITING HEDGEHOG PATHWAY

(71) Applicants: Shivani Shrivastava, Wilmington, DE (US); Rakesh K. Srivastava, Wilmington, DE (US)

(72) Inventors: Shivani SHRIVASTAVA, Wilmington, DE (US); Rakes K. Srivastava, Tyler, TX (US)

(21) Appl. No.: 17/679,073

(22) Filed: Feb. 23, 2022

Related U.S. Application Data
(63) Continuation-in-part of application No. 15/547,069, filed on Jul. 28, 2017, now Pat. No. 11,326,210.
(60) Provisional application No. 62/110,153, filed on Jan. 30, 2015.

(51) Int. Cl. A61K 31/506; A61K 45/06

(52) U.S. Cl. CPC A61K 31/506; A61K 45/06

(57) ABSTRACT

The present invention relates to the use of compositions and methods for treating or preventing a cancer condition in a subject. The use of composition comprises methods of treating or preventing cancer with agents that inhibit Hedgehog pathway activity. The pharmaceutical composition will further comprise agents that inhibit Smoothened receptor and/or Gli transcription in a subject.

This Continuation-in-part application claims the priority benefit of U.S. Non-Provisional application Ser. No. 15/547,069, filed on Jul. 28, 2017, which claims the priority benefits of International Patent Application No. PCT/IB2016/050495, filed on Feb. 1, 2016, which claims the priority benefits of U.S. Provisional Application No. 62/110,153, filed on Jan. 30, 2015.

This patent disclosure contains material that is subject to copyright protection. The copyright owner has no objection to the facsimile reproduction by anyone of the patent document or the patent disclosure as it appears in the U.S. Patent and Trademark Office patent file or records but otherwise reserves any and all copyright rights.

### FIELD OF THE INVENTION

The present invention relates generally to the field of cancer and in particular methods of treating and/or preventing cancer conditions.

More specifically, the present invention relates to the use of compositions for treating and/or preventing a cancer condition in a subject. The use of composition comprises methods of treating or preventing cancer with smoothened and/or Gli inhibitors. The pharmaceutical composition will further comprise agents that inhibit Gli expression or Gli transcriptional activity. Further, the composition comprises methods of using smoothened and/or Gli inhibitors with chemotherapy, immunotherapy, and/or radiotherapy.

### BACKGROUND OF THE INVENTION

Cancer is a condition defined as uncontrolled cell growth, malignant transformation, having cancer stem cell characteristics, and being able to maintain pluripotency, stemness, and resistance to therapy.

Cancer is a deadly disease that can be detected in many ways, including the presence of certain signs and symptoms, screening tests, or medical imaging. Signs of cancer can be found in the blood, plasma, exosomes, body fluids, urine, and tissues.

Deregulation of the hedgehog (Hh) signaling pathway is associated with various malignancies including cancer. The binding of hedgehog ligand to transmembrane Patched (Ptch) receptor results in the withdrawal of inhibitory effects of Patched on smoothened. Thus, the pathway is activated via smoothened through Hh protein stimulation or by the loss of patched activity through Ptch mutations. Activation of the Hh pathway via smoothened induces Gli transcriptional activity. Therefore, targeting of Hh pathway is an attractive strategy for cancer treatment and/or prevention.

One aspect of the present application relates to a method for treating or preventing a cancer condition in a subject, comprising administering to the subject an effective amount of a pharmaceutical composition comprising agents that inhibit hedgehog pathway.

One aspect of the present application relates to a method for treating or preventing a cancer condition in a subject, comprising administering to the subject an effective amount of a pharmaceutical composition comprising agents that inhibit smoothened receptor and/or Gli transcription or expression.

One aspect of the present application relates to a method for treating or preventing a cancer condition in a subject, comprising administering to the subject an effective amount of a pharmaceutical composition comprising agents that inhibit Gli expression and/or Gli transcriptional activity.

Another aspect of the present application relates to the composition of delivering smoothened inhibitors and/or Gli transcription inhibitors in nanoparticles (synthetic or biological materials) conjugated with or without targeting agents and/or imaging agents.

Another aspect of the present application relates to the composition of delivering smoothened inhibitors and/or Gli transcription inhibitors with immunotherapy, and/or surgical intervention.

Another aspect of the present application relates to the method of delivering smoothened inhibitors and/or Gli transcription inhibitors with chemotherapy, immunotherapy, and/or radiotherapy.

### BRIEF DESCRIPTION OF THE DRAWINGS

FIG. 1. Structures of Smoothened and/or Gli inhibitors. Small molecule (synthetic organic, and natural) inhibitors of Smoothened and/or Gli.

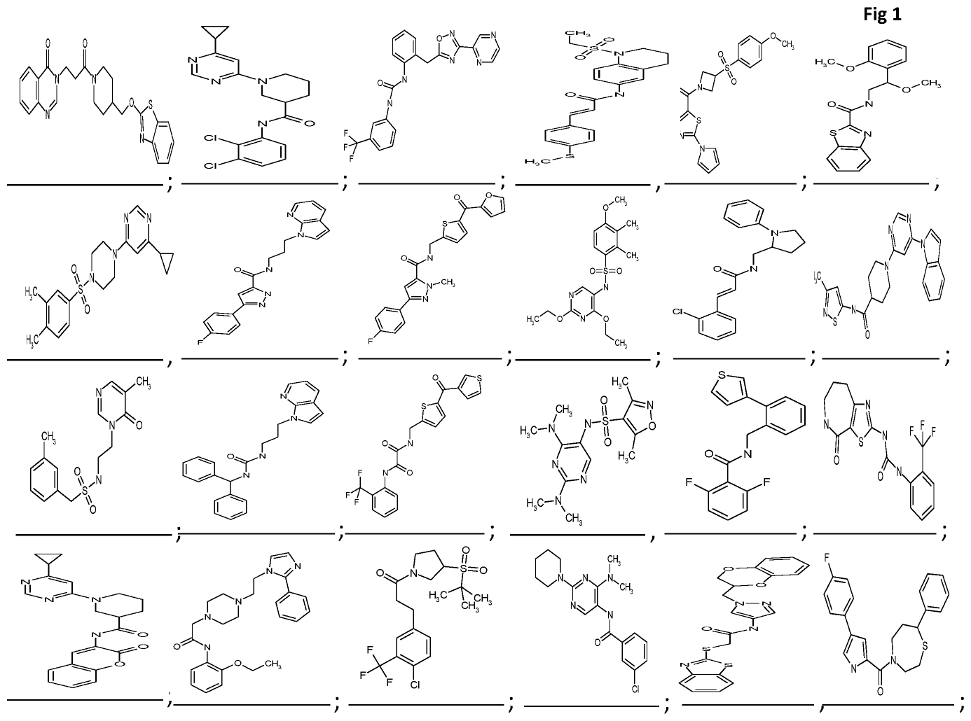

FIG. 2. Structures of Smoothened and/or Gli inhibitors. Small molecule (synthetic organic, and natural) inhibitors of Smoothened and/or Gli.

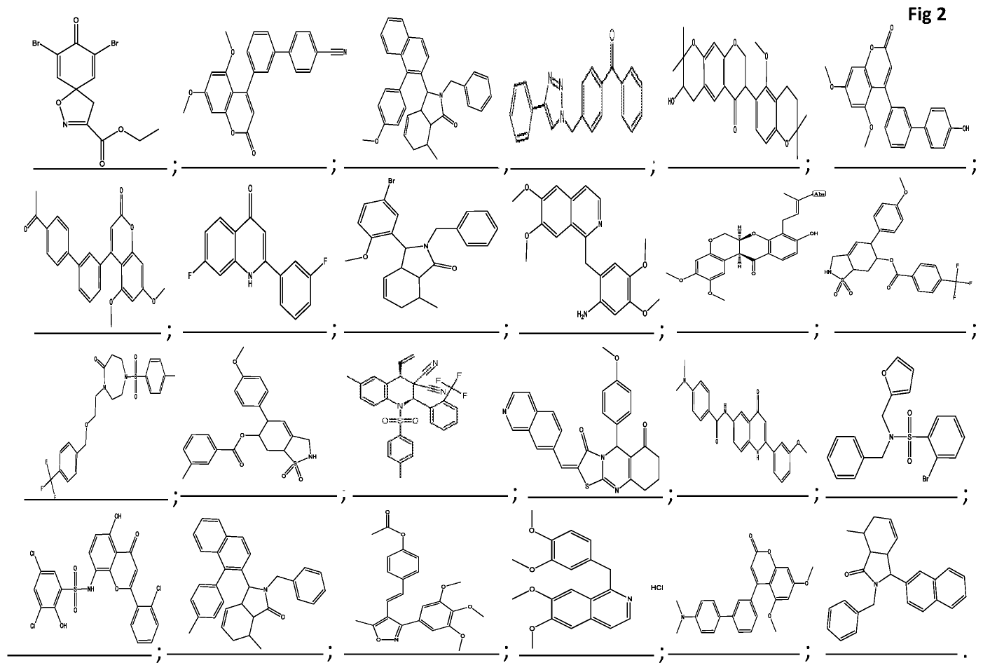

FIG. 3. Effects of Smoothened and/or Gli inhibitors. Smoothened and/or Gli. inhibitors inhibit Gli transcriptional activity, Gli expression, and cancer cell and cancer stem cell proliferation. The doses of Smoothened and/or Gli inhibitors vary from 0.01 μM to 20 μM. Gli transcriptional activity was measured by luciferase assay. Gli expression was measured by qRT-PCR. Cell viability was measured by a steady glow assay.

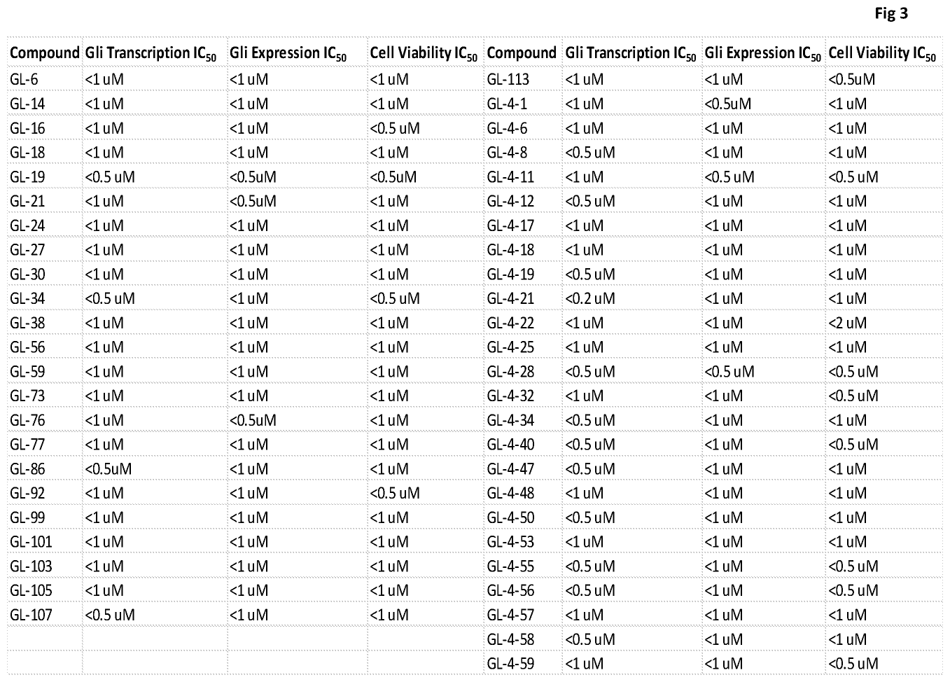

### DETAIL DESCRIPTIONS OF THE INVENTION

All illustrations of the drawings are for the purpose of describing selected versions of the present invention and are not intended to limit the scope of the present invention.

Despite significant advances in diagnosis, surgical techniques, and development of targeted and adjuvant therapies, cancer remains at the epicenter of the current clinical challenges limiting the survival of cancer patients. Therefore, a deeper understanding of the cascade and identification of novel players in the molecular network that could explain differences in the etiology of sporadic cases may serve as a key factor to reduce morbidity and mortality in cancer patients. Efforts aimed at identifying such factors that could be targeted may provide new avenues for cancer treatment and prevention.

The involvement of cancer stem cells (CSCs) or progenitor cells in cancer growth and metastasis has recently been realized. CSCs/progenitor cells appear to be the cause of cancer initiation, progression, and metastasis. CSCs are also a cause of tumor relapse, drug resistance, and chemo- and radiotherapy failure. Stem cells heavily depend on the pluripotency maintaining factors (Nanog, cMyc, Oct-4, Sox-2 and Klf-4) for their self-renewal and survival. Stem cells share many common characteristics with CSCs e.g. expression of cell surface markers and pluripotency maintaining factors. Recent studies demonstrate that the residual population of CSCs after surgery or chemotherapy is responsible for cancer relapse.

The biological activities of CSCs are regulated by several pluripotent transcription factors, such as OCT4, Sox2, Nanog, KLF4, and MYC. In addition, many intracellular signaling pathways, such as Wnt, NF-κB, Notch, Hedgehog, JAK-STAT, PI3K/AKT/mTOR, TGF and PPAR, as well as extracellular factors, such as vascular niches, hypoxia, tumor-associated macrophages, cancer-associated fibroblasts, cancer-associated mesenchymal stem cells, extracellular matrix, and exosomes, have been shown to be very important regulators of CSCs. By altering the expression of genes and pathways by novel agents and approaches, cancers can be prevented and treated by targeting CSCs and progenitor cells. Selective and targeted elimination of the CSCs may be a key for cancer therapy and prevention.

Hedgehog (Hh) signaling pathway is crucially involved in vertebrate development. It is inactive in mature cells of normal adult and found to be aberrantly hyper-activated in cancer and various other malignancies. Evidence suggests that Hh signaling can regulate tissue homeostasis by controlling the production of stem or progenitor cells. Deregulation of the Hh signaling pathway is associated with various malignancies including cancer. The binding of the hedgehog ligand to transmembrane Patched (Ptch) receptor results in the withdrawal of inhibitory effects of Patched on smoothened. Thus, the pathway is activated via smoothened through Hh protein stimulation or by the loss of patched activity through Ptch mutations. Activation of the Hh pathway via smoothened induces Gli transcriptional activity. Therefore, targeting of Hh pathway (e.g. smoothened receptors or Gli transcription) is an attractive strategy for disease treatment and prevention.

The Hh pathway affects neurogenesis and neural patterning during the development of the central nervous system. Dysregulation of the Hh pathway in the brain contributes to aging-related neurodegenerative diseases such as Alzheimer's disease, Parkinson's disease, and amyotrophic lateral sclerosis.

Hedgehog pathway is involved in diverse biological processes such as cell proliferation, differentiation, stemness, epithelial-mesenchymal transition, inflammation, angiogenesis, tumor growth, and metastasis. Due to their pivotal effects on multiple genes and pathways, dysregulated Hh has been reported to be associated with different diseases such as Alzheimer's disease, Parkinson's disease, amyotrophic lateral sclerosis and cancer.

Agents that target cytotoxic T lymphocyte-associated molecule-4 (CTLA-4), programmed cell death receptor-1 (PD-1), and programmed cell death ligand-1 (PD-L1) are the most widely studied and recognized. Immunotherapy also includes molecules such as chimeric monoclonal antibodies and antibody-drug conjugates that target malignant cells and promote their destruction. Genetically modified T cells expressing chimeric antigen receptors are able to recognize specific antigens on cancer cells and subsequently activate the immune system. PD-L1 knockdown reduced expression of several pluripotency-related genes (ALDH1, CD133, OCT4, SOX2, NANOG), impaired cancer stem cell proliferation, and undifferentiated colonies, and decreased the number of cancer stem cells. Gli induces PD-L1 expression. Targeting Gli reverses resistance to PD-1 blockade.

Gli inhibitor reduces the Gli promoter activity. Gli inhibitor can mediate its anti-cancer effects by promoting the proteasomal degradation of Gli transcription factors via inhibition of HSP70/90 chaperone activity.

Some cancers which can be treated by inhibiting cancer stem cells using the compositions and methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); cancers of the lymphomas (hodgkin's disease and non-hodgkin's lymphoma), multiple myeloma, and leukemias (lymphocytic leukemia; myeloid leukemia; monocytic leukemia; and other leukemias).

Other cancers, classified by histological type, that may be treated include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Branchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malignant melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovari, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

In some embodiments, cancer to be treated and the cancer stem cells to be inhibited are from cancers selected from the group consisting of breast cancer, prostate cancer, brain cancer, lung cancer, mesothelioma, melanoma, multiple myeloma, skin cancer, colon cancer, kidney cancer, ovarian cancer, cervical cancer, pancreatic cancer, multiple myeloma, leukemia, and lymphoma.

In some embodiments, Gli expression is inhibited by shRNA, siRNA, oligonucleotides, antisense, microRNA, non-coding RNA, or a combination thereof,

Gene expression levels are determined at the mRNA level (e.g., by RT-PCR, qRT-PCR, QT-PCR oligonucleotide array, etc) or at the protein level (e.g., by Western blot, ELISA, antibody microarray, etc.). Preferred methodologies for determining mRNA expression levels include quantitative reverse transcriptase PCR (QT-PCR), quantitative real-time RT-PCR, oligonucleotide microarray, transcriptome array, microRNA array, gene chip array, methylation array, or combination thereof. Preferred methodologies for determining protein expression levels include the use of ELISAs, Western blotting, and antibody microarrays. The ratios of gene expression and protein expression can also be used.

One aspect of the present application relates to methods for treating or preventing cancer conditions in a subject. In certain embodiments, the method comprises administering to the subject an effective amount of a first agent that inhibits Smoothened receptor and/or Gli transcriptional activity.

In other embodiments, the expression of genes and proteins will be determined in the body fluid, serum, blood, plasma, urine, exosomes and tissue samples.

In other embodiments, Gli activities are defined as their transcriptional activities or protein activities. Inhibitors of Gli by small organic molecules or natural products will inhibit its transcriptional activities and expression, and also Gli-dependent gene transcription. Gli inhibitors will inhibit the growth of cancer stem cells, progenitor cells, cancer cells, angiogenesis, inflammation, epithelial-mesenchymal transition, malignant transformation, cancer growth and metastasis.

In other embodiments, the method further comprises determining the expression of Gli-dependent genes. The status of these genes and proteins may be used, in combination with the inhibitors of Smoothened or Gli for determining the cancer conditions and treatment in the subject.

An aptamer can be chemically linked or conjugated to the above-described nucleic acid inhibitors to form targeted nucleic acid inhibitors (Ray et al., Pharmaceuticals, 3:1761-1778, 2010). An aptamer-siRNA chimera contains a targeting moiety in the form of an aptamer which is linked to an siRNA. In one embodiment, the inhibitor comprises a chimeric aptamer-si RNA oligonucleotide capable of targeting cancer tissues. Preferably, the aptamer is a cell internalizing aptamer. Upon binding to specific cell surface molecules, the aptamer can facilitate internalization into the cell where the nucleic acid inhibitor acts. In one embodiment both the aptamer and the siRNA comprise RNA. The aptamer and the siRNA may comprise any nucleotide modifications as further described herein. In a specific embodiment, the aptamer comprises a targeting moiety such as binding the prostate-specific membrane antigen (PSMA) or mesothelin.

Aptamers can bind very tightly with Kds from the target molecule of less than 10-12 M. Aptamers can bind the target molecule with a very high degree of specificity. For example, aptamers have been isolated that have greater than a 10,000-fold difference in binding affinities between the target molecule and another molecule that differ at only a single position on the molecule.

### Small Organic Molecules

In certain embodiments, Smoothened receptors or Gli activity and/or expression will be inhibited by small organic molecules. Small organic molecules have been successfully used to inhibit the activities of several transcriptional factors which ultimately modulate tumor growth and metastasis. Similarly, small organic molecule inhibitors of Smoothened or Gli will inhibit stemness, epithelial-mesenchymal transition, malignant transformation, cancer growth, angiogenesis, and metastasis.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an,” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “amino” means —NH₂; the term “nitro” means —NO₂; the term “halo” designates —F, —Cl, —Br or —I; the term “mercapto” means —SH; the term “cyano” means —CN; the term “silyl” means —SiH₃, and the term “hydroxy” means —OH.

The term “heteroatom-substituted,” when used to modify a class of organic radicals (e.g. alkyl, aryl, acyl, etc.), means that one, or more than one, hydrogen atom of that radical has been replaced by a heteroatom, or a heteroatom containing group. Examples of heteroatoms and heteroatoms containing groups include: hydroxy, cyano, alkoxy, ═O, ═S, —NO₂, —N(CH₃)₂, amino, or —SH. Specific heteroatom-substituted organic radicals are defined more fully below.

The term “heteroatom-unsubstituted,” when used to modify a class of organic radicals (e.g., alkyl, aryl, acyl, etc.) means that none of the hydrogen atoms of that radical have been replaced with a heteroatom or a heteroatom containing group. Substitution of a hydrogen atom with a carbon atom, or a group consisting of only carbon and hydrogen atoms, is not sufficient to make a group heteroatom substituted. For example, the group —C₆H₄C≡CH is an example of a heteroatom-unsubstituted aryl group, while —C₆H₄F is an example of a heteroatom-substituted aryl group. Specific heteroatom-unsubstituted organic radicals are defined more fully below.

The term “heteroatom-unsubstituted Cₙ-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom unsubstituted C₁-C₁₀-alkyl has 1 to 10 carbon atoms. The term “alkyl” includes straight-chain alkyl groups, branched-chain alkyl groups, cycloalkyl (alicyclic) groups, alkyl heteroatom-substituted cycloalkyl groups, and cycloalkyl heteroatom-substituted alkyl groups. The groups, —CH₃, —CH₂CH₃, —CH₂CH₂CH₃, —CH(CH₃)₂, —CH(CH₂)₂, —CH₂CH₂CH₂CH₃, —CH(CH₃)CH₂CH₃, —CH₂CH(CH₃)₂, —C(CH₃)₃, —CH₂C(CH₃)₃, cyclopentyl, and cyclohexyl, are all examples of heteroatom-unsubstituted alkyl groups.

The term “heteroatom-substituted Cₙ-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom substituted C₁-C₁₀-alkyl has 1 to 10 carbon atoms. The following groups are all examples of heteroatom-substituted alkyl groups: trifluoromethyl, —CH₂F, —CH₂Cl, —CH₂Br, —CH₂OH, —CH₂OCH₃, —CH₂OCH₂CH₃, —CH₂OCH₂CH₂CH₃, —CH₂OCH(CH₃)₂, —CH₂OCH(CH₂)₂, —CH₂OCH₂CF₃, —CH₂OCOCH₃, —CH₂NH₂, —CH₂NHCH₃, —CH₂N(CH₃)₂, —CH₂NHCH₂CH₃, —CH₂N(CH₃)CH₂CH₃, —CH₂NHCH₂CH₂CH₃, —CH₂NHCH(CH₃)₂, —CH₂NHCH(CH₂)₂, —CH₂N(CH₂CH₃)₂, —CH₂CH₂F, —CH₂CH₂Cl, —CH₂CH₂Br, —CH₂CH₂I, —CH₂CH₂OH, CH₂CH₂OCOCH₃, —CH₂CH₂NH₂, —CH₂CH₂N(CH₃)₂, —CH₂CH₂NHCH₂CH₃, —CH₂CH₂N(CH₃)CH₂CH₃, —CH₂CH₂NHCH₂CH₂CH₃, —CH₂CH₂NHCH(CH₃)₂, —CH₂CH₂NHCH(CH₂)₂, —CH₂CH₂N(CH₂CH₃)₂, —CH₂CH₂NHCO₂C(CH₃)₃, and —CH₂Si(CH₃)₃.

The term “heteroatom-unsubstituted Cₙ-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂, —CH═CHCH₃, —CH═CHCH₂CH₃, —CH═CHCH₂CH₂CH₃, —CH═CHCH(CH₃)₂, —CH═CHCH(CH₂)₂, —CH₂CH═CH₂, —CH₂CH═CHCH₃, —CH₂CH═CHCH₂CH₃, —CH₂CH═CHCH₂CH₂CH₃, —CH₂CH═CHCH(CH₃)₂, —CH₂CH═CHCH(CH₂)₂, and —CH═CH—C₆H₅.

The term “heteroatom-substituted Cₙ-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom substituted C₂-C₁₀-alkenyl has 2 to 10 carbon atoms. The groups, —CH═CHF, —CH═CHCl and —CH═CHBr, are examples of heteroatom-substituted alkenyl groups.

The term “heteroatom-unsubstituted Cₙ-alkynyl” refers to a radical, having a linear or branched, cyclic or acyclic structure, further having at least one carbon-carbon triple bond, a total of n carbon atoms, at least one hydrogen atom, and no heteroatoms. For example, a heteroatom-unsubstituted C₂-C₁₀-alkynyl has 2 to 10 carbon atoms. The groups, —C≡CH, —C≡CCH₃, and —C≡CC₆H₅ are examples of heteroatom-unsubstituted alkynyl groups.

The term “heteroatom-substituted Cₙ-alkynyl” refers to a radical, having a single nonaromatic carbon atom as the point of attachment and at least one carbon-carbon triple bond, further having a linear or branched, cyclic or acyclic structure, and having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkynyl has 2 to 10 carbon atoms. The group, —C≡CSi(CH₃)₃, is an example of a heteroatom-substituted alkynyl group.

The term “heteroatom-unsubstituted Cₙ-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom unsubstituted C₆-C₁₀-aryl has 6 to 10 carbon atoms. Examples of heteroatom-unsubstituted aryl groups include phenyl, methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, —C₆H₃(CH₃)CH₂CH₃, —C₆H₄CH═CH₂, —C₆H₄CH═CHCH₃, —C₆H₄C≡CH, —C₆H₄C≡CCH₃, naphthyl, quinolyl, indolyl, and the radical derived from biphenyl. The term “heteroatom-unsubstituted aryl” includes carbocyclic aryl groups, biaryl groups, and radicals derived from polycyclic fused hydrocarbons (PAHs).

The term “heteroatom-substituted Cₙ-aryl” refers to a radical, refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. The term “heteroatom-substituted aryl” includes heteroaryl and heterocyclic aryl groups. It also includes those groups derived from the compounds: pyrrole, furan, thiophene, imidazole, oxazole, isoxazole, thiazole, isothiazole, triazole, pyrazole, pyridine, pyrazine, pyridazine, pyrimidine, and the like. Further examples of heteroatom-substituted aryl groups include the groups: —C₆H₄F, —C₆H₄C₁, —C₆H₄Br, —C₆H₄₁, —C₆H₄OH, —C₆H₄OCH₃, —C₆H₄OCH₂CH₃, —C₆H₄OCOCH₃, —C₆H₄OC₆H₅, —C₆H₄NH₂, —C₆H₄NHCH₃, —C₆H₄NHCH₂CH₃, —C₆H₄CH₂C₁, —C₆H₄CH₂Br, —C₆H₄CH₂OH, —C₆H₄CH₂OCOCH₃, —C₆H₄CH₂NH₂, —C₆H₄N(CH₃)₂, —C₆H₄CH₂CH₂Cl, —C₆H₄CH₂CH₂OH, —C₆H₄CH₂CH₂OCOCH₃, —C₆H₄CH₂CH₂NH₂, —C₆H₄CH₂CH═CH₂, —C₆H₄CF₃, —C₆H₄CN, —C₆H₄C≡CSi(CH₃)₃, —C₆H₄COH, —C₆H₄COCH₃, —C₆H₄COCH₂CH₃, —C₆H₄COCH₂CF₃, —C₆H₄COC₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, and imidazoyl.

The term “heteroatom-unsubstituted Cₙ-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C₇-C₁₀-aralkyl has 7 to 10 carbon atoms. An “aralkyl” includes an alkyl heteroatom-substituted with an aryl group. Examples of heteroatom-unsubstituted aralkyls include phenylmethyl (benzyl) and phenylethyl.

The term “heteroatom-substituted Cₙ-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structure, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms.

The term “heteroatom-unsubstituted Cₙ-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to 10 carbon atoms. The groups, —COH, —COCH₃, —COCH₂CH₃, —COCH₂CH₂CH₃, —COCH(CH₃)₂, —COCH(CH₂)₂, —COC₆H₅, —COC₆H₄CH₃, —COC₆H₄CH₂CH₃, —COC₆H₄CH₂CH₂CH₃, —COC₆H₄CH(CH₃)₂, —COC₆H₄CH(CH₂)₂, and —COC₆H₃(CH₃)₂, are examples of heteroatom-unsubstituted acyl groups.

The term “heteroatom-substituted Cₙ-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The term heteroatom-substituted acyl includes carbamoyl, thiocarboxylate, and thiocarboxylic acid groups. The groups, —COCH₂CF₃, —CO₂H, —CO₂CH₃, —CO₂CH₂CH₃, —CO₂CH₂CH₂CH₃, —CO₂CH(CH₃)₂, —CO₂CH(CH₂)₂, —CONH₂, —CONHCH₃, —CONHCH₂CH₃, —CONHCH₂CH₂CH₃, —CONHCH(CH₃)₂, —CONHCH(CH₂)₂, —CON(CH₃)₂, —CON(CH₂CH₃)CH₃, —CON(CH₂CH₃)₂ and —CONHCH₂CF₃, are examples heteroatom-substituted acyl groups.

The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂.

The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group.

The term “heteroatom-unsubstituted Cₙ-alkenyloxy” refers to a group, having the structure —OR, in which R is a heteroatom-unsubstituted Cₙ-alkenyl, as that term is defined above.

The term “heteroatom-substituted Cₙ-alkenyloxy” refers to a group, having the structure —OR, in which R is a heteroatom-substituted Cₙ-alkenyl, as that term is defined above.

The term “heteroatom-unsubstituted Cₙ-aryloxy” refers to a group, having the structure —OAr, in which Ar is a heteroatom-unsubstituted Cₙ-aryl, as that term is defined above. An example of a heteroatom-unsubstituted aryloxy group is —OC₆H₅.

The term “heteroatom-substituted Cₙ-aryloxy” refers to a group, having the structure —OAr, in which Ar is a heteroatom-substituted Cₙ-aryl, as that term is defined above.

The term “heteroatom-unsubstituted Cₙ-aralkyloxy” refers to a group, having the structure —OAr, in which Ar is a heteroatom-unsubstituted Cₙ-aralkyl, as that term is defined above.

The term “heteroatom-substituted Cₙ-aralkyloxy” refers to a group, having the structure —OAr, in which Ar is a heteroatom-substituted Cₙ-aralkyl, as that term is defined above.

The term “heteroatom-unsubstituted Cₙ-acyloxy” refers to a group, having the structure —OAc, in which Ac is a heteroatom-unsubstituted Cₙ-acyl, as that term is defined above. A heteroatom-unsubstituted acyloxy group includes alkylcarbonyloxy and arylcarbonyloxy groups. For example, —OCOCH₃ is an example of a heteroatom-unsubstituted acyloxy group.

The term “heteroatom-substituted Cₙ-acyloxy” refers to a group, having the structure —OAc, in which Ac is a heteroatom-substituted Cₙ-acyl, as that term is defined above. A heteroatom-substituted acyloxy group includes alkoxycarbonyloxy, aryloxycarbonyloxy, carboxylate, alkylcarbonyl, alkoxycarbonyl, aminocarbonyl, and alkylthiocarbonyl groups.

The term “heteroatom-unsubstituted Cₙ-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cₙ-alkylamino” includes groups, having the structure —NHR, in which R is a heteroatom-unsubstituted Cₙ-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl.

The term “heteroatom-substituted Cₙ-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom substituted C₁-C₁₀-alkylamino has 1 to 10 carbon atoms. The term “heteroatom-substituted Cₙ-alkylamino” includes groups, having the structure —NHR, in which R is a heteroatom-substituted Cₙ-alkyl, as that term is defined above.

The term “heteroatom-unsubstituted Cₙ-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cₙ-alkenylamino” includes groups, having the structure —NHR, in which R is a heteroatom-unsubstituted Cₙ-alkenyl, as that term is defined above. Examples of heteroatom-unsubstituted Cₙ-alkenylamino groups also include dialkenylamino and alkyl(alkenyl)amino groups.

The term “heteroatom-substituted Cₙ-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom substituted C₂-C₁₀-alkenylamino has 2 to 10 carbon atoms. The term “heteroatom-substituted Cₙ-alkenylamino” includes groups, having the structure —NHR, in which R is a heteroatom-substituted Cₙ-alkenyl, as that term is defined above.

The term “heteroatom-unsubstituted Cₙ-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cₙ-alkynylamino” includes groups, having the structure —NHR, in which R is a heteroatom-unsubstituted Cₙ-alkynyl, as that term is defined above. An alkynylamino group includes dialkynylamino and alkyl(alkynyl)amino groups.

The term “heteroatom-substituted Cₙ-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cₙ-alkynylamino” includes groups, having the structure —NHR, in which R is a heteroatom-substituted Cₙ-alkynyl, as that term is defined above.

The term “heteroatom-unsubstituted Cₙ-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom unsubstituted C₆-C₁₀-arylamino has 6 to 10 carbon atoms. The term “heteroatom-unsubstituted Cₙ-arylamino” includes groups, having the structure —NHR, in which R is a heteroatom-unsubstituted Cₙ-aryl, as that term is defined above. A heteroatom-unsubstituted arylamino group includes diarylamino and alkyl(aryl)amino groups.

The term “heteroatom-substituted Cₙ-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom substituted C₆-C₁₀-arylamino has 6 to 10 carbon atoms. The term “heteroatom-substituted Cₙ-arylamino” includes groups, having the structure —NHR, in which R is a heteroatom-substituted Cₙ-aryl, as that term is defined above. A heteroatom-substituted arylamino group includes heteroarylamino groups.

The term “heteroatom-unsubstituted Cₙ-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cₙ-aralkylamino” includes groups, having the structure —NHR, in which R is a heteroatom-unsubstituted Cₙ-aralkyl, as that term is defined above. An aralkylamino group includes diaralkylamino groups.

The term “heteroatom-substituted Cₙ-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cₙ-aralkylamino” includes groups, having the structure —NHR, in which R is a heteroatom-substituted Cₙ-aralkyl, as that term is defined above. The term “heteroatom-substituted aralkylamino” includes the term “heteroaralkylamino.”

The term “heteroatom-unsubstituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cₙ-amido” includes groups, having the structure —NHR, in which R is a heteroatom-unsubstituted Cₙ-acyl, as that term is defined above. The term amido includes N-alkyl-amido, N-aryl-amido, N-aralkyl-amido, acylamino, alkylcarbonylamino, arylcarbonylamino, and ureido groups. The group, —NHCOCH₃, is an example of a heteroatom-unsubstituted amido group.

The term “heteroatom-substituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cₙ-amido” includes groups, having the structure —NHR, in which R is a heteroatom-unsubstituted Cₙ-acyl, as that term is defined above. The group, —NHCO₂CH₃, is an example of a heteroatom-substituted amido group.

The term “heteroatom-unsubstituted Cₙ-alkylthio” refers to a group, having the structure —SR, in which R is a heteroatom-unsubstituted Cₙ-alkyl, as that term is defined above. The group, —SCH₃, is an example of a heteroatom-unsubstituted alkylthio group.

The term “heteroatom-substituted Cₙ-alkylthio” refers to a group, having the structure —SR, in which R is a heteroatom-substituted Cₙ-alkyl, as that term is defined above.

The term “heteroatom-unsubstituted Cₙ-alkenylthio” refers to a group, having the structure —SR, in which R is a heteroatom-unsubstituted Cₙ-alkenyl, as that term is defined above.

The term “heteroatom-substituted Cₙ-alkenylthio” refers to a group, having the structure —SR, in which R is a heteroatom-substituted Cₙ-alkenyl, as that term is defined above.

The term “heteroatom-unsubstituted Cₙ-alkynylthio” refers to a group, having the structure —SR, in which R is a heteroatom-unsubstituted Cₙ-alkynyl, as that term is defined above.

The term “heteroatom-substituted Cₙ-alkynylthio” refers to a group, having the structure —SR, in which R is a heteroatom-substituted Cₙ-alkynyl, as that term is defined above.

The term “heteroatom-unsubstituted Cₙ-arylthio” refers to a group, having the structure —SAr, in which Ar is a heteroatom-unsubstituted Cₙ-aryl, as that term is defined above. The group, —SC₆H₅, is an example of a heteroatom-unsubstituted arylthio group.

The term “heteroatom-substituted Cₙ-arylthio” refers to a group, having the structure —SAr, in which Ar is a heteroatom-substituted Cₙ-aryl, as that term is defined above.

The term “heteroatom-unsubstituted Cₙ-aralkylthio” refers to a group, having the structure —SAr, in which Ar is a heteroatom-unsubstituted Cₙ-aralkyl, as that term is defined above. The group, —SCH₂C₆H₅, is an example of a heteroatom-unsubstituted aralkyl group.

The term “heteroatom-substituted Cₙ-aralkylthio” refers to a group, having the structure —SAr, in which Ar is a heteroatom-substituted Cₙ-aralkyl, as that term is defined above.

The term “heteroatom-unsubstituted Cₙ-acylthio” refers to a group, having the structure —SAc, in which Ac is a heteroatom-unsubstituted Cₙ-acyl, as that term is defined above. The group, —SCOCH₃, is an example of a heteroatom-unsubstituted acylthio group.

The term “heteroatom-substituted Cₙ-acylthio” refers to a group, having the structure —SAc, in which Ac is a heteroatom-substituted Cₙ-acyl, as that term is defined above.

The term “heteroatom-unsubstituted Cₙ-alkylsilyl” refers to a radical, having a single silicon atom as the point of attachment, further having one, two, or three saturated carbon atoms attached to the silicon atom, further having a linear or branched, cyclic or acyclic structure, containing a total of n carbon atoms, all of which are nonaromatic, 5 or more hydrogen atoms, a total of 1 silicon atom, and no additional heteroatoms. For example, a heteroatom-unsubstituted C₁-C₁₀-alkylsilyl has 1 to 10 carbon atoms. An alkylsilyl group includes dialkylamino groups. The groups, —Si(CH₃)₃ and —Si(CH₃)₂C(CH₃)₃, are examples of heteroatom-unsubstituted alkylsilyl groups.

The term “heteroatom-substituted Cₙ-alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S. For example, a heteroatom substituted C₁-C₁₀-alkylsilyl has 1 to 10 carbon atoms.

The small organic molecules can be modified by at least one functional group which includes hydroxyl, methyl, carbonyl, carboxyl, amino, nitro, ether, phosphate, sulhydryl, fluromethyl, ester and carbonyl group.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Examples of inorganic acids which may be used to prepare pharmaceutically acceptable salts include hydrochloric acid, phosphoric acid, sulfuric acid, hydrobromic acid, hydroiodic acid, phosphorous acid and the like. Examples of organic acids which may be used to prepare pharmaceutically acceptable salts include aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like. Other suitable salts are known to one of ordinary skill in the art.

Suitable pharmaceutically acceptable salts may also be formed by reacting the agents of the invention with an organic base such as methylamine, ethylamine, ethanolamine, lysine, ornithine and the like. Other suitable salts are known to one of ordinary skill in the art.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable and as long as the anion or cation does not contribute undesired qualities or effects. Further, additional pharmaceutically acceptable salts are known to those skilled in the art, and may be used within the scope of the invention. Additional examples of pharmaceutically acceptable salts and their methods of preparation and use are presented in Pharmaceutical Salts: Properties, Selection and Use—A Handbook, by C. G. Wermuth and P. H. Stahl, Verlag Helvetica Chimica Acta, 2002, which is incorporated herein by reference.

As used herein, the term “patient” or “subject” is intended to include living organisms. Examples include humans, monkeys, cows, sheep, goats, pigs, horses, dogs, cats, mice, rats, and transgenic species thereof. In a preferred embodiment, the patient is a primate. In an even more preferred embodiment, the primate is a human. Other examples of subjects include experimental animals such as mice, rats, dogs, cats, goats, sheep, pigs, and cows. The experimental animal can be an animal model for a disorder, e.g., a transgenic mouse with an Alzheimer's-type neuropathology. A patient can be a human suffering from a neurodegenerative disease, such as Alzheimer's disease, or Parkinson's disease.

As used herein, the term “IC₅₀” refers to an inhibitory dose which is 50% of the maximum response obtained.

As used herein, the term “water-soluble” means that the compound dissolves in water at least to the extent of 0.010 mole/liter or is classified as soluble according to literature precedence.

As used herein, “predominantly one enantiomer” means that the compound contains at least 85% of one enantiomer, or more preferably at least 90% of one enantiomer, or even more preferably at least 95% of one enantiomer, or most preferably at least 99% of one enantiomer. Similarly, the phrase “substantially free from other optical isomers” means that the composition contains at most 5% of another enantiomer or diastereomer, more preferably 2% of another enantiomer or diastereomer, and most preferably 1% of another enantiomer or diastereomer.

Synthetically produced small organic molecules may incorporate any chemical modifications to the structure that are known to enhance solubility, stability, binding to the target, bioavailability and functionality.

Small organic molecule inhibitors of Smoothened and/or Gli transcription can be delivered in nanoparticles (synthetic or biological materials) conjugated with or without targeting agents and imaging agents. Small organic molecule inhibitors of Smoothened and/or Gli may be delivered using silver nanoparticles, gold nanoparticles, liposomes, micelles, dendrimers, polymers, cellulose, esters, biodegradable particles, and artificial DNA nanostructure.

Small organic molecule inhibitors of Smoothened and/or Gli transcription can be combined with other chemotherapeutic drugs, immunotherapy, and/or irradiation for the treatment and prevention of cancer.

Inhibition of Smoothened Receptor, Gli Expression and/or Activity by Natural Products

Natural molecules from natural sources including plants, microbes, and marine organisms have been the basis of the treatment of human diseases since ancient times. Compounds derived from nature have been important sources of new drugs and also serve as templates for synthetic modification. Many successful anti-cancer drugs currently in use are naturally derived or their analogs and many more are under clinical trials. Natural products have been a rich source of compounds for drug discovery. Natural products are generally non-toxic to humans.

In certain embodiments, Smoothened receptor or Gli transcription activity and/or expression will be inhibited by plant-derived chemicals. These plant-derived chemicals may comprise of pure chemical/compound or a mixture of chemicals. Natural products have successfully been used to inhibit cancer cell proliferation, tumor growth, angiogenesis, and metastasis. Similarly, natural products either pure or complex, will inhibit Gli activity and/or expression in cancer cells. Natural product inhibitors of Smoothened and/or Gli will inhibit stemness, epithelial-mesenchymal transition, malignant transformation, angiogenesis, cancer growth, and metastasis.

Natural products either isolated from plants or synthetically produced may incorporate any chemical modifications to the structure that are known to enhance solubility, stability, binding to target, bioavailability and functionality.

Natural product inhibitors of Smoothened and/or Gli will be delivered in nanoparticles (synthetic or biological materials) conjugated with or without targeting agents or imaging agents. Natural product inhibitors of Smoothened and/or Gli can be delivered using silver nanoparticles, gold nanoparticles, liposomes, micelles, dendrimers, polymers, cellulose, esters, biodegradable particles, and artificial DNA nanostructure.

Natural product inhibitors of Smoothened and/or Gli can be combined with other chemotherapeutic drugs, immunotherapy, irradiation and/or imaging agent for the treatment and prevention of cancer.

### Administration of Smoothened and Gli Transcription Inhibitors

The compounds of the present invention may be administered, e.g., orally, topically or by injection (e.g. subcutaneous, intravenous, intraperitoneal, etc.) Depending on the route of administration, the active compound may be coated in a material to protect the compound from the action of acids and other natural conditions which may inactivate the compound. In the case of cancer therapy, the agents may be administered intra-tumorally, circumferential to a tumor mass, locally to the tumor vasculature or lymphatic system, regionally or systemically. They may also be administered to a resected tumor bed, for example, by syringing or by a post-operative catheter with continuous perfusion/infusion.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984).

The therapeutic compound may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water-soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard- or soft-shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

Compounds of the invention may also be formulated for local administration, e.g., for topical administration to the skin or mucosa, for topical administration to the eye, for delivery to the lungs by inhalation, or by incorporation into a biocompatible matrix for controlled release to a specified site over an extended period of time (e.g., as an active ingredient in a drug-eluting cardiac stent). In certain cases, significant systemic concentrations may also be achieved by these routes of administration (e.g., via pulmonary or transmucosal delivery).

Active compounds are administered at a therapeutically effective dosage sufficient to treat a condition associated with a condition in a patient. A “therapeutically effective amount” preferably reduces the number of symptoms of the condition in the infected patient by at least about 20%, more preferably by at least about 40%, even more preferably by at least about 60%, and still more preferably by at least about 80% relative to untreated subjects. For example, the efficacy of a compound can be evaluated in an animal model system that may be predictive of efficacy in treating the disease in humans, such as the model systems shown in the examples and drawings.

By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These may be targeted to a particular cell type via antibodies, receptors, or receptor ligands. Vehicles such as “stealth” and other antibody conjugated liposomes (including lipid mediated drug targeting to colonic carcinoma), receptor-mediated targeting of DNA through cell-specific ligands, lymphocyte directed tumor targeting, and highly specific therapeutic retroviral targeting of murine glioma cells in vivo. In general, receptors are involved in pathways of endocytosis, either constitutive or ligand-induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

The compositions described herein can be packaged together in any suitable combination as a kit useful for performing or aiding in the performance of, the disclosed method. In some embodiments, the kit for treating cancer conditions comprises an inhibitor of Gli expression or activity. The inhibitors or activators may comprise any of the described bioactive components.

A composition disclosed herein may be administered in a number of ways depending on whether local or systemic treatment is desired, and on the area to be treated. For example, the compositions may be administered orally, parenterally (e.g., intravenous, subcutaneous, intravesical, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

The exact amount of the compositions required will vary from subject to subject, depending on the species, age, weight and general condition of the subject, the particular nucleic acid or vector used, its mode of administration and the like. Guidance can be found in the literature for appropriate dosages for given classes of pharmaceutical products. For example, a typical daily dosage of the disclosed composition used alone might range from about 1 μg/kg to up to 100 mg/kg of body weight or more per day, depending on the factors mentioned above. The monitoring Gli expression or transcriptional activity levels can be used to predict drug response or resistance, as well as identify patients who may be candidates for anti-Hh therapy. The term “anti-Hh therapy” refers to methods for inhibiting Gli expression or Gli activity.

In some embodiments, the agents described herein are combined with one or more conventional chemotherapeutic agents. Exemplary chemotherapeutic agents for use in the present invention include 5-alpha-reductase inhibitors, including finasteride, dutasteride, turosteride, bexlosteride, izonsteride, FCE 28260, and SKF 105, 111; integrin-linked kinase (ILK) inhibitors, such as QLT-0267; secreted frizzled-related protein-1 (sFRP1), secreted frizzled-related protein-2 (sFRP2), secreted frizzled related protein-3 (sFRP3/1-RZB), secreted frizzled-related protein-4 (sFRP4), secreted frizzled-related protein-5 (SFRP5), Dickkopf-1 (DKK1), Dickkopf-2 (DKK2), Dickkopf-3 (DKK3), Wnt inhibitory factor-1 (WIF1), cerberus, sclerostin, IWR-1-endo, IWP-2, IWP-3, IWP4, pyrvinium, XAV939, and other WNT signalling pathway inhibitors; bevacizumab (Avastin), cabazitaxel, ketoconazole, prednisone, Sipuleucel-T (APC8015, Provenge), Alpharadin (radium-223 chloride), MDV3100, orteronel (TAK-700), PROSTVAC, cabozantinib (XL-184), DMAPT; cyclopamine, vismodegib, and other hedgehog (Hh) signalling pathway inhibitors; Notch pathway inhibitors; JAK/STAT pathway inhibitors; NF-kB pathway inhibitors; PI3K/Akt pathway inhibitors; flutamide, luprolide, antiestrogens, such as tamoxifen; antimetabolites and cytotoxic agents, such as daunorubicin, flourouracil, floxuridine, interferon alpha, methotrexate, plicamycin, mecaptopurine, thioguanine, adramycin, carmustine, lomustine, cytarabine, cyclophosphamide, doxorubicin, estramustine, altretamine, hydroxyurea, ifosfamide, procarbazine, mutamycin, busulfan, mitoxantrone, carboplatin, cisplatin, streptozocin, bleomycin, dactinomycin, idamycin, hormones such as, medroxyprogesterone, estramustine, ethinyl oestradiol, oestradiol, leuprolide, megestrol, octreotide, diethylstilbestrol, chlorotrianisene, etoposide, podophyllotoxin, goserelin, nitrogen mustard derivatives such as, melphalan, chlorambucil, methlorethamine, thiotepa, steroids such as, betamethasone, and other antineoplastic agents such as live Mycobacterium bovis, dicarbazine, asparaginase, leucovoribn, mitotane, vincristine, vinblastine, texotere, cydophosphamide, adriamycin, 5-flourouracil, hexamethylmelamine, acivicin; aclarubicin; acodazole hydrochloride; acrqnine; adozolesin; aldesloukin; altretamine; ambomycin; ametantrone acetate; am inogluthim ide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomyrin; edatrexate; eflomithine hydrochloride; elsamitrucin; enloplatin; enprorfate; epipropidine; epirubicin hydrochloride; erbulozole; esorubicin hydrochloride; estramustine; estramustine phosphate sodium; etanidazole; ethiodized oil; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; gold Au 198; hydroxyurea; idarubicin hydrochloride; ifosfamide; ilmofosine; interferon alfa-2a; interferon alfa-2b; interferon alfa-n1; interferon alfa-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; strontium chloride Sr 89; sulofenur; talisomycin; taxane; taxoid; tecogalan sodium; tegafur; teloxantrone hydrochloride; temoporfin; teniposide; teroxirono; testolactone; thiamiprine; thioguanine; thiotepa; tiazofurin; tirapazamine; topotecan hydrochloride; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, 20-epi-1,25 dihydroxyvitamin D3; 5-ethynyluracil; abiraterone, aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; DHEA; bromineepiandrosterone; epiandrosterone; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTSA, arginine deaminase; asulacrine; atamestane; atrimustine; axinastatin 1; axinastatin 2; axinastatin 3; azasetron; azatoxin; azatyrosine; baccatin III derivatives; balanol; batimastat, BCR/ABL antagonists; benzochlorins; benzoylstaursporine; beta lactam derivatives; beta-alethine; betaclamycin B; betulinic acid; bFGF inhibitor, bicalutamide; bisantrene; bisazindinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthrequinones; cycloplatam; cypemycin; cytarabine ocfosfate; cytolytic factor; cytostatin; dacliximab; decitabine; dehydrodidemnin B; deslorelin; dexifostamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarbine; fenretinido; filgrastim; frnasteride; flavopiridol; flezelastine; fluasterone; fludarabine; fluorodaunorunicin hydrochloride; torfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists, interferons; interleukins; iobonguane; iododoxorubicin; ipomeanol, 4; trinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole liarozole; linear polyamine analogue; lipophilicadisaccharide peptide; lipophilic platinum compounds; lissoclinamide-7; lobaplatin, lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosplioryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzam ides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulator; nitroxide antioxidant; nitrullyn; O6-benzylguanine; octreotide; okicenone; oligonucleotides; onapristone; orldarisetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaepergase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum comprex; platinum compounds; platinum-triamine coil iplex; porfimer sodium; portiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purino mucleoside phosphorylast inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitor; ras-GAP inhibitor, retalliptine demethylated; rhenium Re186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim, Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmonine; superactive vasoactive intestinal peptide antagonist; suradista; suram in; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erytrocyte gene therapy; velaresol; venom, anti-venom, veramine; verdins; verteporfin; vinorelbine; vinxaltine; vitaxin; vorozole; zanoterone; zeniplatin; zilascorb; zinostatin stimalamer, immunostimulating drugs or therapeutic agents, their metabolites, salts and derivatives thereof, and combinations thereof.

In some embodiments, these agents can be used in conjunction with other cancer therapies. In some embodiments, one or more of the compounds are used with other anticancer drugs, such as, for example, gemcitabine and lapatinib, irradiation to sensitize cancer stem cells, and/or surgical intervention. Other anticancer drugs that can be combined with the compounds as described herein include, for example, Abraxane, Aldara, Alimta, Aprepitant, Arimidex, Aromasin, Arranon, Arsenic Trioxide, Avastin, Bevacizumab, Bexarotene, Bortezomib, Cetuximab, Clofarabine, Clofarex, Clolar, Dacogen, Dasatinib, Ellence, Eloxatin, Emend, Erlotinib, Faslodex, Femara, Fulvestrant, Gefitinib, Gemtuzumab Ozogamicin, Gemzar, Gleevec, Herceptin, Hycamtin, Imatinib Mesylate, Iressa, Kepivance, Lenalidomide, Levulan, Methazolastone, Mylosar, Mylotarg, Nanoparticle Paclitaxel, Nelarabine, Nexavar, Nolvadex, Oncaspar, Oxaliplatin, Paclitaxel, Paclitaxel Albumin-stabilized Nanoparticle Formulation, Palifermin, Panitumumab, Pegaspargase, Pemetrexed Disodium, Platinol-AQ, Platinol, Revlim id, Rituxan, Sclerosol Intrapleural Aerosol, Sorafenib Tosylate, Sprycel, Sunitinib Malate, Sutent, Synovir, Tamoxifen, Tarceva, Targretin, Taxol, Taxotere, Temodar, Temozolomide, Thalomid, Thalidomide, Topotecan Hydrochloride, Trastuzumab, Trisenox, Vectibix, Velcade, Vidaza, Vorinostat, Xeloda, Zoledronic Acid, Zolinza, Zometa, doxorubicin, adriamycin, bleomycin, daunorubicin, dactinomycin, epirubicin, idarubicin, mitoxantrone, valrubicin, hydroxyurea, mitomycin, fluorouracil, 5-FU, methotrexate, floxuridine, interferon alpha-2b, glutamic acid, plicamycin, 6-thioguanine, am inopterin, pemetrexed, raltitrexed, cladribine, clofarabine, fludarabine, mercaptopurine, pentostatin, capecitabine, cytarabine, carmustine, BCNU, lomustine, CCNU, cytosine arabinoside, cyclophosphamide, estramustine, hydroxyurea, procarbazine, mitomycin, busulfan, medroxyprogesterone, estramustine phosphate sodium, ethinyl estradiol, estradiol, megestrol acetate, methyltestosterone, diethylstilbestrol diphosphate, chlorotrianisene, testolactone, mephalen, mechlorethamine, chlorambucil, chlormethine, ifosfamide, bethamethasone sodium phosphate, dicarbazine, asparaginase, mitotane, vincristine, vinblastine, etoposide, teniposide, Topotecan, IFN-gamma, irinotecan, campto, irinotecan analogs, carmustine, fotemustine, lomustine, streptozocin, carboplatin, oxaliplatin, BBR3464, busulfan, dacarbazine, mechlorethamine, procarbazine, thioTEPA, uramustine, vindesine, vinorelbine, alemtuzumab, tositumomab, methyl am inolevulinate, porfimer, verteporfin, lapatinib, nilotinib, vandetanib, ZD6474, alitretinoin, altretamine, amsacrine, anagrelide, denileukin diftitox, estramustine, hydroxycarbamide, masoprocol, mitotane, tretinoin, or other anticancer drugs, including, for example, antibiotic derivatives, cytotoxic agents, angiogenesis inhibitors, hormones or hormone derivatives, nitrogen mustards and derivatives, steroids and combinations, and antimetbolites. Other chemotherapeutic drugs include Notch inhibitor, TGFβ inhibitor, Pdx1 inhibitor, Oct4 inhibitor, Sox2 inhibitor, Sox4 inhibitor, KLF4 inhibitor, TCF/LEF inhibitor, Nanog inhibitor, AKT inhibitor, FLT3 kinase inhibitor, PI3 Kinase inhibitor, PI3 kinase/mTOR (dual inhibitor), PI3K/AKT pathway inhibitor, Hedgehog pathway inhibitor, Gli inhibitor, JAK/STAT pathway inhibitor, Ras/MEK/ERK pathway inhibitor, and BRAF inhibitor. In further particular aspects of the invention, an anticancer drug comprises two or more of the foregoing anticancer drugs.

In some embodiments, these agents can be used in conjunction with immunotherapies. In some embodiments, one or more of the compounds are used with immunotherapy, and/or surgical intervention. Immunotherapies include, but not limited to, immune checkpoint inhibitors, T-cell transfer therapy, monoclonal antibodies, treatment vaccines, and immune system modulators.

Suitable compositions and dosage forms also include tablets, capsules, caplets, gel caps, troches, dispersions, suspensions, solutions, syrups, transdermal patches, gels, powders, magmas, lozenges, creams, pastes, plasters, lotions, discs, suppositories, liquid sprays for nasal or oral administration, dry powder or aerosolized formulations for inhalation, and the like.

Oral dosage forms are preferred for those therapeutic agents that are orally active, and include tablets, capsules, caplets, solutions, suspensions and/or syrups, and may also comprise a plurality of granules, beads, powders or pellets that may or may not be encapsulated. Such dosage forms can be prepared using conventional methods known to those in the field of pharmaceutical formulation and described in the pertinent texts, e.g., in Remington: The Science and Practice of Pharmacy, 20th Edition, Gennaro, A. R., Ed. (Lippincott, Williams and Wilkins, 2000).

Tablets and capsules represent the most convenient oral dosage forms, in which case solid pharmaceutical carriers are employed. Tablets may be manufactured using standard tablet processing procedures and equipment. One method for forming tablets is by direct compression of a powdered, crystalline, or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In addition to the active agent(s), tablets prepared for oral administration will generally contain other materials such as binders, diluents, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methylcellulose, ethylcellulose, hydroxyethylcellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch, and powdered sugar. Lubricants are used to facilitate tablet manufacture; examples of suitable lubricants include, for example, magnesium stearate, calcium stearate, and stearic acid. Stearates, if present, preferably represent at no more than approximately 2 wt. % of the drug-containing core. Disintegrants are used to facilitate disintegration of the tablet, and are generally starches, clays, celluloses, algins, gums or crosslinked polymers. Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilizers are used to inhibit or retard drug decomposition reactions that include, by way of example, oxidative reactions. Surfactants may be anionic, cationic, amphoteric or nonionic surface-active agents.

The dosage form may also be a capsule, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals. If the active agent-containing composition is present within the capsule in liquid form, a liquid carrier is necessary to dissolve the active agent(s). The carrier must be compatible with the capsule material and all components of the pharmaceutical composition and must be suitable for ingestion.

Solid dosage forms, whether tablets, capsules, caplets, or particulates, may, if desired, be coated so as to provide for delayed release. Dosage forms with delayed release coatings may be manufactured using standard coating procedures and equipment. Such procedures are known to those skilled in the art and described in the pertinent texts, e.g., in Remington, supra. Generally, after preparation of the solid dosage form, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained release dosage forms provide for drug release over an extended time period and may or may not be delayed release. Generally, as will be appreciated by those of ordinary skill in the art, sustained release dosage forms are formulated by dispersing a drug within a matrix of a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

Parenteral administration, if used, is generally characterized by injection, including intramuscular, intraperitoneal, intravenous (IV) and subcutaneous injection. Injectable formulations can be prepared in conventional forms, either as liquid solutions or suspensions, solid forms suitable for solution or suspension in liquid prior to injection, or as emulsions. In some embodiments, sterile injectable suspensions are formulated according to techniques known in the art using suitable dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. In some embodiments, the formulation for parenteral administration is a controlled release formulation, such as delayed or sustained release.

Any of the active agents may be administered in the form of a salt, ester, amide, prodrug, active metabolite, derivative, or the like, provided that the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base using conventional methodology, and involves reaction with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are salts prepared with organic acids. Conversely, preparation of basic salts of acid moieties that may be present on an active agent are prepared in a similar manner using a pharmaceutically acceptable base such as sodium hydroxide, potassium hydroxide, ammonium hydroxide, calcium hydroxide, trimethylamine, or the like. Preparation of esters involves functionalization of hydroxyl and/or carboxyl groups that may be present within the molecular structure of the drug. The esters are typically acyl-substituted derivatives of free alcohol groups, i.e., moieties that are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

Other derivatives and analogs of the active agents may be prepared using standard techniques known to those skilled in the art of synthetic organic chemistry or may be deduced by reference to the pertinent literature. In addition, chiral active agents may be in isomerically pure form, or they may be administered as a racemic mixture of isomers.

In some embodiments, drugs are given to a subject in nanoparticles with targeting agents with or without imaging agents. Imaging agents include, but are not limited to, magnetic iron oxide, quantum dots, PET, single-photon emission tomography, and optical imaging including fluorescence-mediated tomography and near-infrared fluorescence reflectance (NIRF) imaging, fluorescent agents, ¹⁸F-labeled fluorodeoxyglucose, and radionucleotide. The medical imaging modalities include magnetic resonance imaging, computed tomography, positron emission tomography, single-photon emission computerized tomography, optical imaging, ultrasound, and photoacoustic imaging.

Targeted drug delivery, sometimes called smart drug delivery, is a method of delivering medication to a patient in a manner that increases the concentration of the medication in some parts of the body relative to others. The goal of a targeted drug delivery system is to prolong, localize, target and have a protected drug interaction with the diseased tissue. The conventional drug delivery system is the absorption of the drug across a biological membrane, whereas the targeted release system is when the drug is released in a dosage form. The advantages to the targeted release system are the reduction in the frequency of the dosages taken by the patient, having a more uniform effect of the drug, reduction of drug side effects, and reduced fluctuation in circulating drug levels. Drugs can be delivered using nanoparticles, liposomes, micelles, dendrimers, polymers, cellulose, biodegradable particles, and artificial DNA nanostructure. Particles (diameter 80 to 600 nM) comprised of the polymer poly(lactic-co-glycolic acid) (PLGA) are widely studied as therapeutic delivery vehicles because they are biodegradable and biocompatible.

1. A method for treating or preventing cancer in a subject, comprising administering to the subject a pharmaceutical composition comprising an effective amount of at least one agent that inhibits at least one of Smoothened receptor, or Gli transcriptional activity, wherein
Gli is highly expressed, and an agent is a small molecule represented by a compound having the chemical structure of:

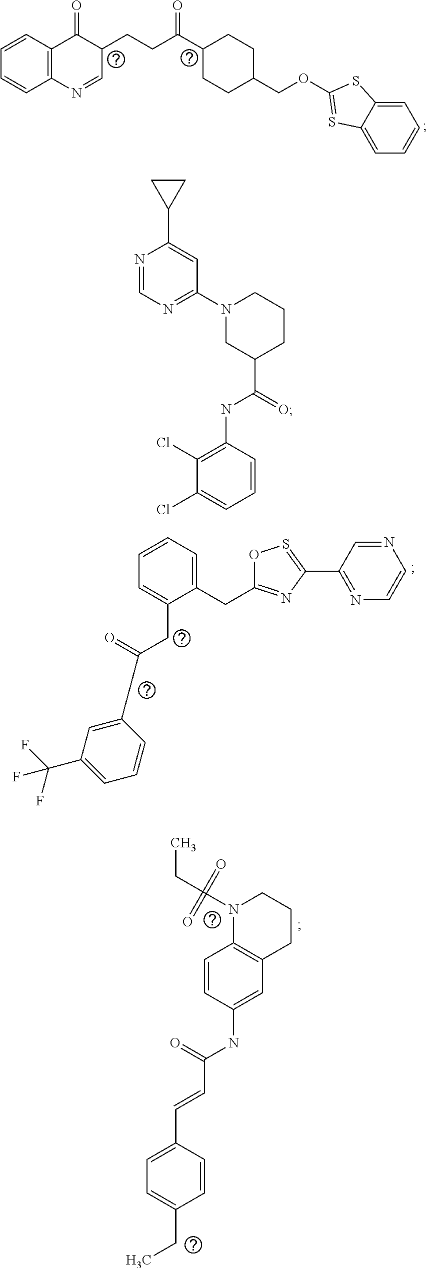

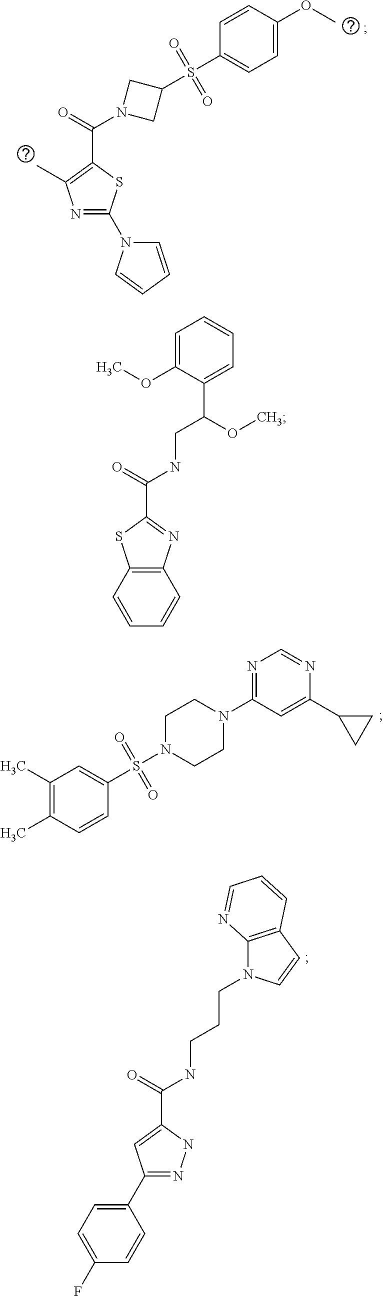

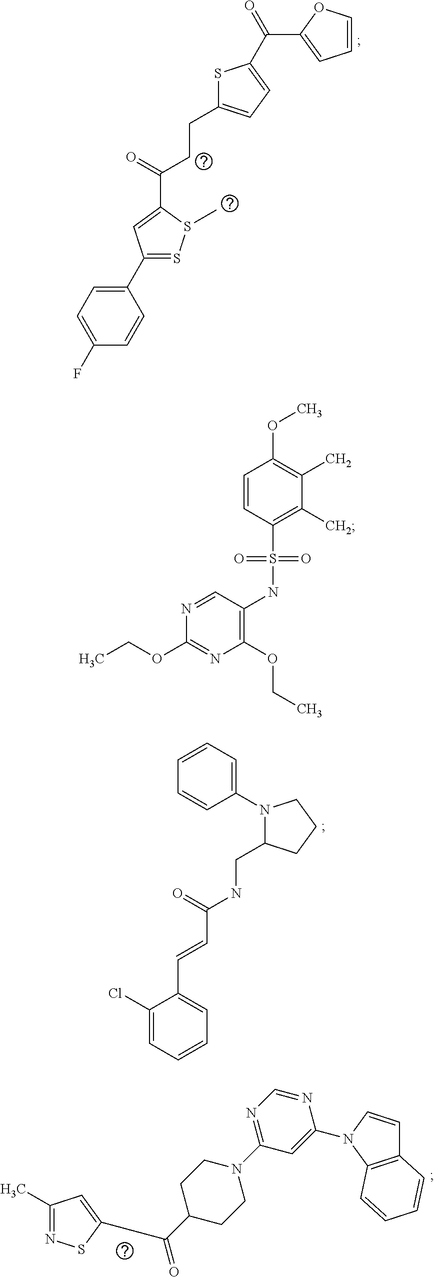

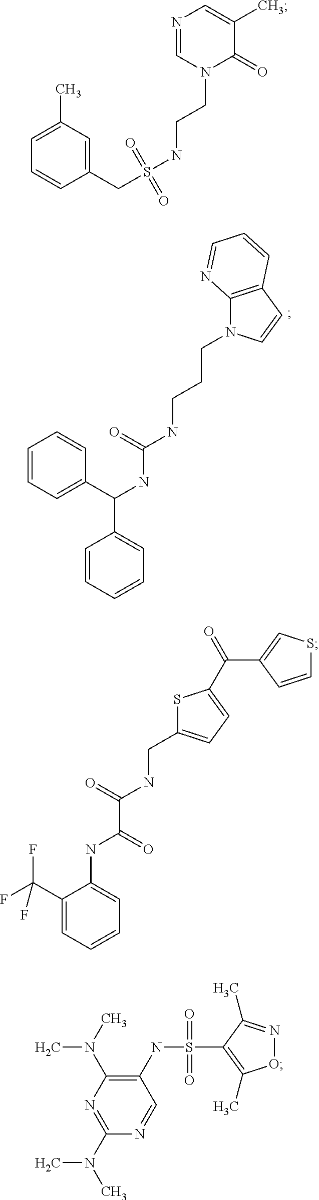 [SMILES: Cc1cccc(CS(=O)(=O)NCCn2cncc(C)c2=O)c1 ; O=C(NCCCn1ccc2cccnc21)NC(c1ccccc1)c1ccccc1 ; O=C(NCc1ccc(C(=O)c2ccsc2)s1)C(=O)Nc1ccccc1C(F)(F)F ; Cc1noc(C)c1S(=O)(=O)Nc1cnc(N(C)C)nc1N(C)C]

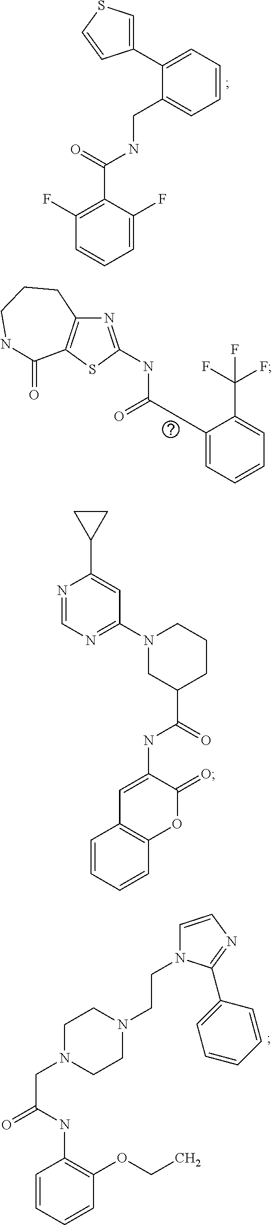

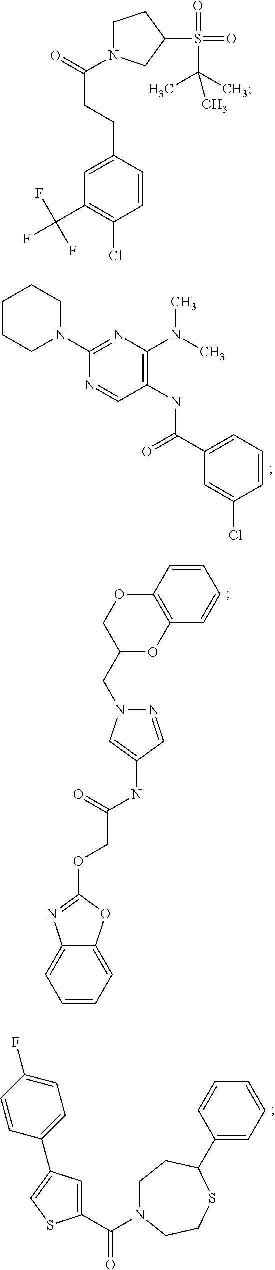

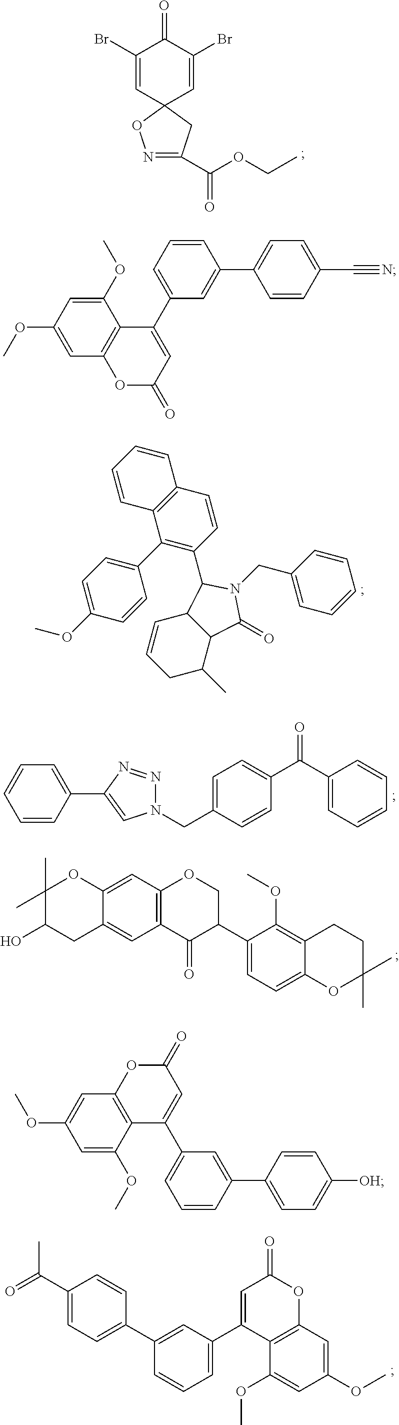

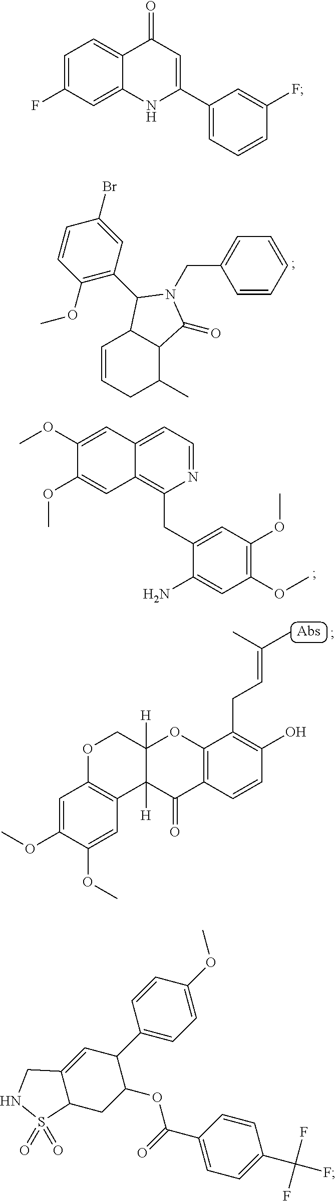

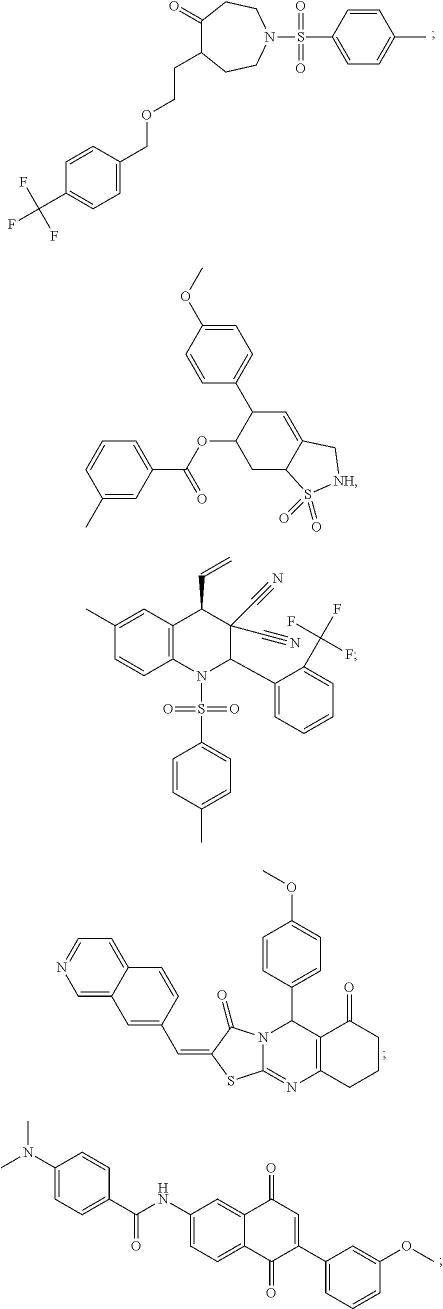

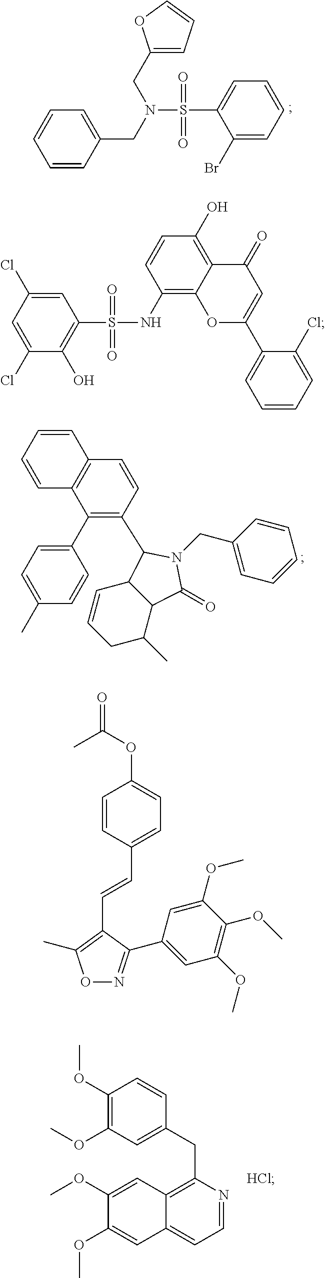

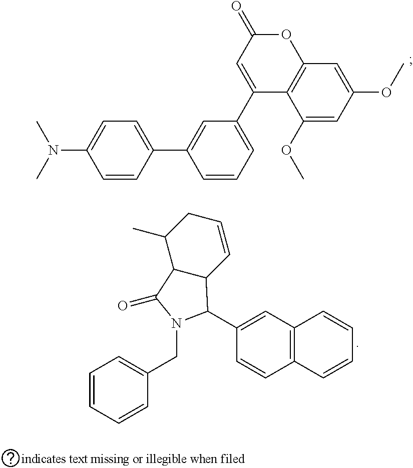

2. The composition of claim 1, wherein said agent comprises a targeting moiety capable of binding to the surface of a cancer cell, wherein said targeting moiety is selected from the group consisting of aptamers, peptides, biodegradable materials, antibody-derived epitope binding domains, cellular ligands, and combination thereof.

3. The method of claim 1, wherein the method further comprises:
determining Gli expression or Gli transcription activity of the said subject.

4. The composition of claim 1, wherein at least one agent inhibits cancer cell or cancer stem cell growth.

5. The composition of claim 1, wherein at least one agent is administered in nanoparticles containing an imaging agent with or without a targeting agent.

6. The composition of claim 1, wherein at least one agent is administered to the subject in combination with one or more chemotherapeutic drugs, immunotherapy or radiation for treatment or prevention of cancer.

7. The composition as claimed in claim 1, wherein said agent can be modified by at least one functional group which includes hydroxyl, methyl, carbonyl, carboxyl, amino, nitro, ether, phosphate, sulhydryl, fluromethyl, ester and carbonyl group.

8. The composition of claim 7, wherein said agent comprises a targeting moiety capable of binding to the surface of a cancer cell, wherein said targeting moiety is selected from the group consisting of aptamers, peptides, biodegradable materials, antibody-derived epitope binding domains, cellular ligands, and combination thereof.

9. The method of claim 7, wherein the method further comprises:
determining Gli expression or Gli transcription activity of the said subject.

10. The composition of claim 7, wherein at least one agent inhibits cancer cell or cancer stem cell growth.

11. The composition of claim 7, wherein at least one agent is administered in nanoparticles containing an imaging agent with or without a targeting agent.

12. The composition of claim 7, wherein at least one agent is administered to the subject in combination with one or more chemotherapeutic drugs, immunotherapy or radiation for treatment or prevention of cancer.
